(19) United States
(12) Patent Application Publication
(10) Pub. No.: US 2025/0381321 A1
(43) Pub. Date: Dec. 18, 2025

(54) SUPRAMOLECULAR POLYMER PUTTY FOR BONE/TISSUE REGENERATION

(71) Applicant: AMPHIX BIO, INC., Chicago, IL (US)

(72) Inventors: Nicholas A. Sather, Chicago, IL (US); Charlotte H. Chen, Chicago, IL (US)

(21) Appl. No.: 19/103,600

(22) PCT Filed: Aug. 17, 2023
(86) PCT No.: PCT/US2023/072387

§ 371 Date: Feb. 13, 2025

Related U.S. Application Data
(60) Provisional application No. 63/399,467, filed on Aug. 19, 2022.

(51) Int. Cl. A61L 27/48; A61L 27/44; A61L 27/54

(52) U.S. Cl. CPC A61L 27/48; A61L 27/446; A61L 27/54; A61L 2430/02

(57) ABSTRACT

Spinal fusion is a surgical procedure to treat debilitating back and neck pain where two adjacent spinal vertebrae are fused by inducing bone growth between them. While iliac crest autograft bone has been the gold standard for spinal fusions due to its low cost and reliable fusion results, harvest site morbidity and limited volume have led to a search for alternatives. Provided herein are compositions comprising peptide amphiphiles, soft covalent polymers, and ceramic materials. Composite putty-like materials are provided for medical uses, in particular for the repair of bone/tissue injuries/defects and the regeneration of bone or other tissue.

### CROSS-REFERENCE TO RELATED APPLICATION

The present invention claims the benefit of U.S. Provisional Patent Application Ser. No. 63/399,467, filed Aug. 19, 2022, which is hereby incorporated by reference in its entirety.

### SEQUENCE LISTING

The text of the computer readable sequence listing filed herewith, titled “41195-601_SEQUENCE_LISTING”, created Aug. 17, 2023, having a file size of 31,731 bytes, is hereby incorporated by reference in its entirety.

### FIELD

Provided herein are compositions comprising peptide amphiphiles, soft covalent polymers, and ceramic materials. Composite putty-like materials are provided for medical uses, in particular for the repair of bone/tissue injuries/defects and the regeneration of bone or other tissue.

### BACKGROUND

Spinal fusion is a surgical procedure to treat debilitating back and neck pain where two adjacent spinal vertebrae are fused by inducing bone growth between them. While iliac crest autograft bone has been the gold standard for spinal fusions due to its low cost and reliable fusion results, harvest site morbidity and limited volume have led to a search for alternatives. Spinal biologics such as bone morphogenetic protein-2 (BMP-2) are popular among surgeons because they offer a synthetic, off-the-shelf implant requiring a single surgical site and have consistent clinical outcomes. However, major safety concerns about supraphysiological doses of BMP-2 have limited its approval for use only in the lumbar region of the spine, and high manufacturing costs of recombinant proteins have been prohibitive for adoption by some hospitals/institutions.

### SUMMARY

Provided herein are compositions comprising peptide amphiphiles, soft covalent polymers, and ceramic materials. Composite putty-like materials are provided for medical uses, in particular for the repair of bone/tissue injuries/defects and the regeneration of bone or other tissue.

In some embodiments, provided herein are compositions comprising a composite material comprising: (a) peptide amphiphile nanofibers; (b) a polymer component; and (c) a ceramic component.

In some embodiments, the peptide amphiphile nanofibers comprise a supramolecular assembly of peptide amphiphiles, the peptide amphiphiles comprising: (i) a hydrophobic non-peptidic segment; (ii) a structural peptide segment; and (iii) a charged segment. In some embodiments, the polymer component comprises polyethylene glycol (PEG).

In some embodiments, the polymer component comprises a PEG selected from PEG 200, PEG 300, PEG 400, PEG 500, PEG 550, PEG 600, PEG 700, PEG 800, PEG 900, PEG 1000, PEG 1450, PEG 3350, PEG 4500, PEG 8000 or combinations thereof. In some embodiments, the polymer component comprises PEG 600 and PEG 1450.

In some embodiments, the ceramic component comprises hydroxyapatite (HA), tricalcium phosphate (TCP), bioglass, calcium sulfate. In some embodiments, the ceramic component comprises TCP. In some embodiments, the composite material further comprises a bioactive factor.

In some embodiments, the composite material comprises <5 wt % of the peptide amphiphile nanofibers, 60-80 wt % of the polymer component, and 20-40 wt % of the ceramic component. In some embodiments, the composite material comprises <1 wt % of the peptide amphiphile nanofibers, 65-75 wt % of the polymer component, and 25-35 wt % of the ceramic component. In some embodiments, the composite material comprises <1 wt % of the peptide amphiphile nanofibers, about 69 wt % of the polymer component, and about 30 wt % of the ceramic component.

In some embodiments, all or a portion of the peptide amphiphiles further comprise a bioactive peptide segment. In some embodiments, the bioactive peptide segment mimics the biological function of or is capable of binding to a bioactive factor and/or cellular component. In some embodiments, the bioactive factor and/or cellular component is selected from bone morphogenic proteins, transforming growth factors, epidermal growth factor, growth differentiation factors, human endothelial cell growth factor, granulocyte macrophage colony stimulating factor, nerve growth factor, vascular endothelial growth factor, fibroblast growth factor, insulin-like growth factor, cartilage derived morphogenetic protein, platelet rich plasma, platelet derived growth factor, insulin growth factor one, and platelet-derived growth factor.

In some embodiments, the peptide amphiphile nanofibers comprise a supramolecular assembly of: (i) bioactive peptide amphiphiles comprising (A) a hydrophobic non-peptidic segment. (B) a structural peptide segment, (C) a charged segment, and (D) a bioactive peptide; and (ii) diluent peptide amphiphiles comprising (A) a hydrophobic non-peptidic segment, (B) a structural peptide segment, and (C) a charged segment. In some embodiments, (i) and (ii) are present at a ratio between 1:10 and 10:1. In some embodiments, (i) and (ii) are present are present at a ratio between 1:2 and 2:1. In some embodiments, the hydrophobic non-peptidic segment comprises an acyl chain. In some embodiments, the acyl chain comprises C₆-C₂₀. In some embodiments, the hydrophobic non-peptidic segment of the diluent peptide amphiphile and the hydrophobic non-peptidic segment of the bioactive peptide amphiphile the same length. In some embodiments, the hydrophobic non-peptidic segment of the diluent peptide amphiphile and the hydrophobic non-peptidic segment of the bioactive peptide amphiphile are different lengths. In some embodiments, the hydrophobic non-peptidic segment of the diluent peptide amphiphile is C₁₆ and the hydrophobic non-peptidic segment of the bioactive peptide amphiphile is C₁₂. In some embodiments, the structural peptide segment is an alanine- and valine-rich peptide segment. In some embodiments, the alanine- and valine-rich peptide segment comprises AAVV (SEQ ID NO: 2), AAAVVV (SEQ ID NO: 3) VVAA (SEQ ID NO: 4), or VVVAAA (SEQ ID NO: 5). In some embodiments, the charged peptide segment is a glutamate- and/or aspartate-rich segment. In some embodiments, the glutamate- and/or aspartate-rich segment comprises 2-7 amino acids in length with 50% or more amino acids selected from Glu (E) and/or Asp (D) residues. In some embodiments, the glutamate- and/or aspartate-rich segment comprises EE or EEE. In some embodiments, the bioactive peptide is a BMP-2 binding peptide. In some embodiments, the bioactive peptide comprises at least 50% sequence identity with TSPHVPYGGGS (SEQ ID NO: 1). In some embodiments, the binding sequence comprises TSPHVPYGGGS (SEQ ID NO:1).

In some embodiments, provided herein are methods comprising administering a composite material described herein to a subject. In some embodiments, the composite material is administered to repair a bone or tissue injury or defect.

In some embodiments, provided herein is a composition comprising: (a) peptide amphiphile nanofibers comprising a supramolecular assembly of: (i) bioactive peptide amphiphiles comprising (A) a hydrophobic non-peptidic segment, (B) a structural peptide segment. (C) a charged segment, and (D) a BMP-2 binding peptide; and (ii) diluent peptide amphiphiles comprising (A) a hydrophobic non-peptidic segment, (B) a structural peptide segment, and (C) a charged segment; (b) a PEG-containing polymer component; (c) a ceramic component comprising TCP or HA; and (d) BMP-2.

In some embodiments, provided herein is a composition comprising: (a) peptide amphiphile nanofibers comprising a supramolecular assembly of: (i) bioactive peptide amphiphiles comprising (A) a hydrophobic non-peptidic segment, (B) a structural peptide segment. (C) a charged segment, and (D) a BMP-2 binding peptide; and (ii) diluent peptide amphiphiles comprising (A) a hydrophobic non-peptidic segment, (B) a structural peptide segment, and (C) a charged segment; (b) a PEG-containing polymer component; and (c) a ceramic component comprising TCP or HA; wherein the composition does not comprise BMP-2.

In some embodiments, provided herein are methods of promoting osteogenesis comprising administering to a subject a composite material described herein.

In some embodiments, provided herein are methods of repairing a bone injury or defect in a subject comprising administering a composite material described herein.

In some embodiments, provided herein are methods of promoting arthrodesis comprising administering to a subject a composite material described herein.

In some embodiments, provided herein are methods of promoting spinal fusion comprising administering to a subject a composite material described herein.

### BRIEF DESCRIPTION OF THE DRAWINGS

FIG. 1. Chemical structures of PA molecules comprising supramolecular polymer to bind and potentiate the signal of rhBMP-2 protein: Diluent PA (top) and Bioactive PA (bottom).

FIG. 2. Storage modulus of different carriers for bioactive supramolecular polymer (all containing supramolecular polymer). The collagen carrier is described in US20170106120A1 (incorporated by reference in its entirety), while the putty carriers are described herein.

FIG. 3. Rat spinal fusion rate for putties with supramolecular polymer, with small or large TCP particles as indicated, without and with rhBMP-2 as indicated. Rat spines are scored by three blinded reviewers, with 0 meaning no fusion, 1 meaning one side of the spine fused, and 2 meaning both sides are fused. Animals with an average score 1 or higher are considered successfully fused for the fusion rate calculation.

FIG. 4. Storage modulus of different putties containing supramolecular polymer, with indicated weight percentages of large TCP particles (250-1000 μm).

FIG. 5. Rat spinal fusion scores and rate for putties with supramolecular polymer, with 10% or 30% TCP by weight as indicated. The scores are the average of three blinded reviewers, with 0 meaning no fusion, 1 meaning one side of the spine fused, and 2 meaning both sides are fused. Animals with an average score 1 or higher are considered fused for the fusion rate calculation.

FIG. 6A-C. Characterization of PA putty. (A) Fourier transform infrared spectrographs (FTIR) of putty containing PA. The peak around 1630 cm-1 indicates the presence of β-sheeted structures. (B) Confocal micrographs of PA putty where the PEG was tagged with fluorescein (FITC), the PA tagged with rhodamine (TAMRA), and bovine serum albumin tagged with Alexa Fluor 647 (far red dye). The individual channels and overlay are plotted. (C). Enzyme-linked immunosorbent assay (ELISA) showing BMP-2 release from a collagen ACS carrier (current clinical product) and from the putty. All materials were loaded with 100 ng of BMP-2 to start FIG. 7. Rabbit spinal fusion rate for putties with supramolecular polymer, with indicated BMP-2 doses. Rabbit spines are scored by three blinded reviewers, with 0 meaning no fusion, 1 meaning one side of the spine fused, and 2 meaning both sides are fused. Animals with an average score 1 or higher are considered successfully fused for the fusion rate calculation.

### Definitions

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “a peptide amphiphile” is a reference to one or more peptide amphiphiles and equivalents thereof known to those skilled in the art, and so forth.

The term “amino acid” refers to natural amino acids, unnatural amino acids, and amino acid analogs, all in their D and L stereoisomers, unless otherwise indicated, if their structures allow such stereoisomeric forms. Embodiments herein refer to various amino acid abbreviations (single-letter or three-letter abbreviations) that will be understood by those in the field. Any amino acid abbreviations not defined herein refer to their field-accepted meaning.

The term “proteinogenic amino acids” refers to the 20 amino acids coded for in the human genetic code, and includes alanine (Ala or A), arginine (Arg or R), asparagine (Asn or N), aspartic acid (Asp or D), cysteine (Cys or C), glutamine (Gln or Q), glutamic acid (Glu or E), glycine (Gly or G), histidine (His or H), isoleucine (Ile or I), leucine (Leu or L). Lysine (Lys or K), methionine (Met or M), phenylalanine (Phe or F), proline (Pro or P), serine (Ser or S), threonine (Thr or T), tryptophan (Trp or W), tyrosine (Tyr or Y) and valine (Val or V). Selenocysteine and pyroolysine may also be considered proteinogenic amino acids

The term “non-proteinogenic amino acid” refers to an amino acid that is not naturally-encoded or found in the genetic code, and is not incorporated biosynthetically into proteins during translation. Non-proteinogenic amino acids may be “unnatural amino acids” (amino acids that do not occur in nature) or “naturally-occurring non-proteinogenic amino acids” (e.g., norvaline, ornithine, homocysteine, etc.). Examples of non-proteinogenic amino acids include, but are not limited to, azetidinecarboxylic acid, 2-aminoadipic acid, 3-aminoadipic acid, beta-alanine, naphthylalanine, aminopropionic acid, 2-aminobutyric acid, 4-aminobutyric acid, 6-aminocaproic acid, 2-aminoheptanoic acid, 2-aminoisobutyric acid, 3-aminoisbutyric acid, 2-aminopimelic acid, tertiary-butylglycine, 2,4-diaminoisobutyric acid, desmosine, 2,2′-diaminopimelic acid, 2,3-diaminopropionic acid, N-ethylglycine, N-ethylasparagine, homoproline, hydroxylysine, allo-hydroxylysine, 3-hydroxyproline, 4-hydroxyproline, isodesmosine, allo-isoleucine, N-methylalanine, N-alkylglycine including N-methylglycine. N-methylisoleucine, N-alkylpentylglycine including N-methylpentylglycine. N-methylvaline, naphthylalanine, norvaline, norleucine (“Norleu”), octylglycine, ornithine, pentylglycine, pipecolic acid, thioproline, homolysine, and homoarginine. Non-proteinogenic also include D-amino acid forms of any of the amino acids herein, as well as non-alpha amino acid forms of any of the amino acids herein (beta-amino acids, gamma-amino acids, delta-amino acids, etc.), all of which are in the scope herein and may be included in peptides herein.

The term “amino acid analog” refers to a natural or unnatural amino acid where one or more of the C-terminal carboxy group, the N-terminal amino group and side-chain functional group has been chemically blocked, reversibly or irreversibly, or otherwise modified to another functional group. For example, aspartic acid-(beta-methyl ester) is an amino acid analog of aspartic acid; N-ethylglycine is an amino acid analog of glycine; or alanine carboxamide is an amino acid analog of alanine. Other amino acid analogs include methionine sulfoxide, methionine sulfone. S-(carboxymethyl)-cysteine. S-(carboxymethyl)-cysteine sulfoxide and S-(carboxymethyl)-cysteine sulfone.

As used herein, the term “peptide” refers a short polymer of amino acids linked together by peptide bonds. In contrast to other amino acid polymers (e.g., proteins, polypeptides, etc.), peptides are of about 50 amino acids or less in length. A peptide may comprise natural amino acids, non-natural amino acids, amino acid analogs, and/or modified amino acids. A peptide may be a subsequence of naturally occurring protein or a non-natural (artificial) sequence.

As used herein, the term “artificial” refers to compositions and systems that are designed or prepared by a human, and are not naturally occurring. For example, an artificial peptide or nucleic acid is one comprising a non-natural sequence (e.g., a peptide without 100% identity with a naturally-occurring protein or a fragment thereof).

As used herein, a “conservative” amino acid substitution refers to the substitution of an amino acid in a peptide or polypeptide with another amino acid having similar chemical properties, such as size or charge. For purposes of the present disclosure, each of the following eight groups contains amino acids that are conservative substitutions for one another: 1) Alanine (A) and Glycine (G); 2) Aspartic acid (D) and Glutamic acid (E); 3) Asparagine (N) and Glutamine (Q); 4) Arginine (R) and Lysine (K); 5) Isoleucine (I). Leucine (L), Methionine (M), and Valine (V); 6) Phenylalanine (F), Tyrosine (Y), and Tryptophan (W); 7) Serine(S) and Threonine (T); and 8) Cysteine (C) and Methionine (M).

Naturally occurring residues may be divided into classes based on common side chain properties, for example: polar positive (histidine (H), lysine (K), and arginine (R)); polar negative (aspartic acid (D), glutamic acid (E)); polar neutral (serine(S), threonine (T), asparagine (N), glutamine (Q)); non-polar aliphatic (alanine (A), valine (V), leucine (L), isoleucine (I), methionine (M)); non-polar aromatic (phenylalanine (F), tyrosine (Y), tryptophan (W)); proline and glycine; and cysteine. As used herein, a “semi-conservative” amino acid substitution refers to the substitution of an amino acid in a peptide or polypeptide with another amino acid within the same class.

In some embodiments, unless otherwise specified, a conservative or semi-conservative amino acid substitution may also encompass non-naturally occurring amino acid residues that have similar chemical properties to the natural residue. These non-natural residues are typically incorporated by chemical peptide synthesis rather than by synthesis in biological systems. These include, but are not limited to, peptidomimetics and other reversed or inverted forms of amino acid moieties. Embodiments herein may, in some embodiments, be limited to natural amino acids, non-natural amino acids, and/or amino acid analogs.

Non-conservative substitutions may involve the exchange of a member of one class for a member from another class.

As used herein, the term “sequence identity” refers to the degree to which two polymer sequences (e.g., peptide, polypeptide, nucleic acid, etc.) have the same sequential composition of monomer subunits. The term “sequence similarity” refers to the degree with which two polymer sequences (e.g., peptide, polypeptide, nucleic acid, etc.) differ only by conservative and/or semi-conservative amino acid substitutions. The “percent sequence identity” (or “percent sequence similarity”) is calculated by: (1) comparing two optimally aligned sequences over a window of comparison (e.g., the length of the longer sequence, the length of the shorter sequence, a specified window, etc.), (2) determining the number of positions containing identical (or similar) monomers (e.g., same amino acids occurs in both sequences, similar amino acid occurs in both sequences) to yield the number of matched positions, (3) dividing the number of matched positions by the total number of positions in the comparison window (e.g., the length of the longer sequence, the length of the shorter sequence, a specified window), and (4) multiplying the result by 100 to yield the percent sequence identity or percent sequence similarity. For example, if peptides A and B are both 20 amino acids in length and have identical amino acids at all but 1 position, then peptide A and peptide B have 95% sequence identity. If the amino acids at the non-identical position shared the same biophysical characteristics (e.g., both were acidic), then peptide A and peptide B would have 100% sequence similarity. As another example, if peptide C is 20 amino acids in length and peptide D is 15 amino acids in length, and 14 out of 15 amino acids in peptide D are identical to those of a portion of peptide C, then peptides C and D have 70% sequence identity, but peptide D has 93.3% sequence identity to an optimal comparison window of peptide C. For the purpose of calculating “percent sequence identity” (or “percent sequence similarity”) herein, any gaps in aligned sequences are treated as mismatches at that position.

Any peptides described herein as having a particular percent sequence identity or similarity (e.g., at least 70%) with a reference sequence, may also be expressed as having a maximum number of substitutions (or terminal deletions) with respect to that reference sequence. For example, a sequence “having at least 70% sequence identity with SEQ ID NO:X” may have up to 3 substitutions relative to SEQ ID NO:X (when SEQ ID NO: X is 10 amino acids in length), and may therefore also be expressed as “having 3 or fewer substitutions relative to SEQ ID NO: X.” Further, a sequence “having at least 80% sequence similarity with SEQ ID NO:X” may have 0, 1, or 2 non-conservative substitutions relative to SEQ ID NO:X, and may therefore also be expressed as “having 2 or fewer non-conservative substitutions relative to SEQ ID NO: X.”

As used herein, the term “nanofiber” refers to an elongated or threadlike filament (e.g., having a significantly greater length dimension that width or diameter) with a diameter typically less than 100 nanometers (e.g., 10 nm).

As used herein, the term “supramolecular” (e.g., “supramolecular complex,” “supramolecular interactions,” “supramolecular fiber,” “supramolecular polymer,” etc.) refers to the non-covalent interactions between molecules (e.g., polymers, marcomolecules, etc.) and the multicomponent assemblies, complexes, systems, and/or fibers that form as a result.

As used herein, the term “physiological conditions” refers to the range of conditions of temperature. pH and tonicity (or osmolality) normally encountered within tissues in the body of a living human.

As used herein, the terms “self-assemble” and “self-assembly” refer to formation of a discrete, non-random, aggregate structure from component parts; said assembly occurring spontaneously through random movements of the components (e.g. molecules) due only to the inherent chemical or structural properties and attractive forces of those components.

As used herein, the term “peptide amphiphile” refers to a molecule that, at a minimum, includes a non-peptide lipophilic (hydrophobic) segment, a structural peptide segment and optionally a functional peptide segment. The peptide amphiphile may express a net charge at physiological pH, either a net positive or negative net charge, or may be zwitterionic (i.e., carrying both positive and negative charges). Certain peptide amphiphiles consist of or comprise: (1) a hydrophobic, non-peptidic segment (e.g., comprising an acyl group of six or more carbons), (2) a structural or β-sheet-forming peptide segment; (3) a carboxyl-rich peptide segment, and (4) a bioactive moiety (e.g., BMP-2 binding moiety).

As used herein and in the appended claims, the term “lipophilic moiety” or “hydrophobic moiety” refers to the moiety disposed on the N-terminus of the peptide amphiphile (e.g., an acyl moiety), and may be herein and elsewhere referred to as the lipophilic or hydrophobic segment or component. The hydrophobic component should be of a sufficient length to provide amphiphilic behavior and micelle (or nanosphere or nanofiber) formation in water or another polar solvent system. Accordingly, in the context of the embodiments described herein, the hydrophobic component preferably comprises a single, linear acyl chain of the formula: Cₙ₋₁H₂ₙ₋₁C(O)— where n=6-22. In some embodiments, a linear acyl chain is the lipophilic group, palmitic acid. However, other small lipophilic groups may be used in place of the acyl chain.

As used herein, the term “structural peptide” or “beta-sheet forming peptide” refers to the intermediate amino acid sequence of the peptide amphiphile molecule between the hydrophobic segment and the charged peptide segment of the peptide amphiphile. This “structural peptide” or “beta-sheet forming peptide” is generally composed of three to ten amino acid residues with non-polar, uncharged side chains, selected for their propensity to form a beta-sheet secondary structure. Examples of suitable amino acid residues selected from the twenty naturally occurring amino acids include Met (M), Val (V), Ile (I), Cys (C), Tyr (Y), Phe (F), Gln (Q), Leu (L), Thr (T), Ala (A), and Gly (G) (listed in order of their propensity to form beta sheets). However, non-naturally occurring amino acids of similar beta-sheet forming propensity may also be used. Peptide segments capable of interacting to form beta sheets and/or with a propensity to form beta sheets are understood (See, e.g., Mayo et al. Protein Science (1996), 5:1301-1315; herein incorporated by reference in its entirety). In a preferred embodiment, the N-terminus of the structural peptide segment is covalently attached to the oxygen of the lipophilic segment and the C-terminus of the structural peptide segment is covalently attached to the N-terminus of the charged peptide segment.

As used herein, the terms “carboxy-rich peptide segment,” “acidic peptide segment.” and “negatively charged peptide segment” refer to the peptide sequence that is either (i) intermediately disposed between the structural peptide segment (beta-sheet forming segment) and the bioactive peptide (BMP-2 binding segment), or (ii) the C-terminal segment of a PA without a bioactive peptide (e.g., a diluent PA). In some embodiments, the carboxy-rich peptide segment two or more amino acid residues that have side chains displaying carboxylic acid side chains (e.g., Glu (E), Asp (D), or non-natural amino acids). A carboxy-rich peptide segment may optionally contain one or more additional (e.g., non-acidic) amino acid residues. Non-natural amino acid residues with acidic side chains could be used, as will be evident to one ordinarily skilled in the art. There may be from about 2 to about 7 amino acids, and or about 3 or 4 amino acids in this segment.

As used herein, the term “bioactive peptide” refers to amino acid sequences that mediate the action of sequences, molecules, or supramolecular complexes associated therewith. Peptide amphiphiles and structures (e.g., nanofibers) bearing bioactive peptides (e.g., BMP-2 binding peptides) exhibits the functionality of the functional peptide.

### DETAILED DESCRIPTION

Provided herein are compositions comprising peptide amphiphiles, soft covalent polymers, and ceramic materials. Composite putty-like materials are provided for medical uses, in particular for the repair of bone/tissue injuries/defects and the regeneration of bone or other tissue. In some embodiments, composition further comprise a bioactive factor (e.g., capable of being bound by a bioactive peptide of the peptide amphiphile). In other embodiments, the peptide amphiphile comprises a bioactive peptide capable of binding a bioactive factor (e.g., BMP-2), but the composition does not comprise exogenous bioactive factor (e.g., the composition is configured to interact with endogenous bioactive factor (e.g., BMP-2) upon administration to a treatment site.

In some embodiments, the composite materials herein comprise a peptide amphiphile nanofiber component. Peptide amphiphile molecules have been demonstrated to be useful as building blocks to create biomaterials, for example, in regenerative medicine. PAs are designed to self-assemble in aqueous conditions into high-aspect-ratio nanofibers t measuring approximately 10 nanometers in diameter and microns in length. Their formation is driven mainly by secondary interactions such as collapse of hydrophobic molecular segments away from an aqueous environment and hydrogen bonding among peptide segments leading to .beta.-sheet secondary structure (Hartgerink, E. Beniash, S. Stupp, Science 2001, 294, 1684; incorporated by reference in their entireties). These supramolecular nanofibers can be designed to display a high surface density of bioactive peptides capable of diverse functions. Various PA nanofibers have been demonstrated to be useful in repair of the central nervous system and cartilage, neovascularization of ischemic heart tissue, enamel growth, and bone repair, among others (Tysseling-Mattiace et al. Journal of Neuroscience 2008, 28, 3814; Shah et al. Proceedings of the National Academy of Sciences 2010, 107, 3293; Webber et al. Proceedings of the National Academy of Sciences 2011, 108, 13438; Huang et al. Biomaterials 2010, 31, 9202; Mata et al. Biomaterials 2010, 31, 6004; Sargeant et al/Biomaterials 2008, 29, 161; incorporated by reference in their entireties).

In some embodiments, the peptide amphiphile molecules and compositions of the embodiments described herein are synthesized using preparatory techniques well-known to those skilled in the art, preferably, by standard solid-phase peptide synthesis, with the addition of a fatty acid in place of a standard amino acid at the N-terminus (or C-terminus) of the peptide, in order to create the lipophilic segment. Synthesis typically starts from the C-terminus, to which amino acids are sequentially added using either a Rink amide resin (resulting in an —NH2 group at the C-terminus of the peptide after cleavage from the resin), or a Wang resin (resulting in an —OH group at the C-terminus). Accordingly, embodiments described herein encompasses peptide amphiphiles having a C-terminal moiety that may be selected from the group consisting of —H, —OH, —COOH, —CONH2, and —NH2.

In some embodiments, peptide amphiphiles comprise a hydrophobic (non-peptide) segment linked to a peptide. In some embodiments, the peptide comprises a structural segment (e.g., hydrogen-bond-forming segment, beta-sheet-forming segment, etc.), and a charged segment (e.g., acidic segment, basic segment, zwitterionic segment, etc.). In some embodiments, the peptide further comprises linker or spacer segments for adding solubility, flexibility, distance between segments, etc. In some embodiments, peptide amphiphiles comprise a spacer segment (e.g., peptide and/or non-peptide spacer) at the opposite terminus of the peptide from the hydrophobic segment. In some embodiments, the spacer segment comprises peptide and/or non-peptide elements. In some embodiments, the spacer segment comprises one or more active functional groups (e.g., alkene, alkyne, azide, thiol, etc.). In some embodiments, various segments may be connected by linker segments (e.g., peptide (e.g., GG) or non-peptide (e.g., alkyl, OEG, PEG, etc.) linkers).

The lipophilic or hydrophobic segment is typically incorporated at the N- or C-terminus of the peptide after the last amino acid coupling, and is composed of a fatty acid or other acid that is linked to the N- or C-terminal amino acid through an acyl bond. In aqueous solutions, PA molecules self-assemble (e.g., into cylindrical micelles (a.k.a., nanofibers)) that bury the lipophilic segment in their core and display the bioactive peptide on the surface. The structural peptide undergoes intermolecular hydrogen bonding to form beta sheets that orient parallel to the long axis of the micelle.

In some embodiments, compositions described herein comprise PA building blocks that in turn comprise a hydrophobic segment and a peptide segment. In certain embodiments, a hydrophobic (e.g., hydrocarbon and/or alkyl/alkenyl/alkynyl tail, or steroid such as cholesterol) segment of sufficient length (e.g., 2 carbons, 3 carbons, 4 carbons, 5 carbons, 6 carbons, 7 carbons, 8 carbons, 9 carbons, 10 carbons, 11 carbons, 12 carbons, 13 carbons, 14 carbons, 15 carbons, 16 carbons, 17 carbons, 18 carbons, 19 carbons, 20 carbons, 21 carbons, 22 carbons, 23 carbons, 24 carbons, 25 carbons, 26 carbons, 27 carbons, 28 carbons, 29 carbons, 30 carbons or more, or any ranges there between) is covalently coupled to peptide segment (e.g., a peptide comprising a segment having a preference for beta-strand conformations or other supramolecular interactions) to yield a peptide amphiphile molecule. In some embodiments, a plurality of such PAs will self-assemble in water (or aqueous solution) into a nanostructure (e.g., nanofiber). In various embodiments, the relative lengths of the peptide segment and hydrophobic segment result in differing PA molecular shape and nanostructural architecture. For example, a broader peptide segment and narrower hydrophobic segment results in a generally conical molecular shape that has an effect on the assembly of PAs (See, e.g., J. N. Israelachvili Intermolecular and surface forces; 2nd ed.; Academic: London San Diego, 1992; herein incorporated by reference in its entirety). Other molecular shapes have similar effects on assembly and nanostructural architecture.

In some embodiments, to induce self-assembly of an aqueous solution of peptide amphiphiles, the pH of the solution may be changed (raised or lowered) or multivalent ions, such as calcium, or charged polymers or other macromolecules may be added to the solution.

In some embodiments, the hydrophobic segment is a non-peptide segment (e.g., alkyl/alkenyl/alkynyl group). In some embodiments, the hydrophobic segment comprises an alkyl chain (e.g., saturated) of 4-25 carbons (e.g., 4, 5, 6, 7, 8, 9, 10, 11, 12, 13, 14, 15, 16, 17, 18, 19, 20, 21, 22, 23, 24, 25), fluorinated segments, fluorinated alkyl tails, heterocyclic rings, aromatic segments, pi-conjugated segments, cycloalkyls, oligothiophenes etc. In some embodiments, the hydrophobic segment comprises an acyl/ether chain (e.g., saturated) of 2-30 carbons (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30).

In some embodiments, PAs comprise one or more peptide segments. Peptide segments may comprise natural amino acids, modified amino acids, unnatural amino acids, amino acid analogs, peptidomimetics, or combinations thereof. In some embodiments, peptide segments comprise at least 50% (e.g., 55%, 60%, 65%, 70%, 75%, 80%, 85%, 90%, 95%, 100%, or ranges therebetween) sequence identity or similarity (e.g., conservative or semi-conservative) to one or more of the peptide sequences described herein.

In some embodiments, peptide amphiphiles comprise a charged peptide segment. The charged segment may be acidic, basic, or zwitterionic.

In some embodiments, peptide amphiphiles comprise an acidic peptide segment. For example, in some embodiments, the acidic peptide comprises one or more (e.g., 1, 2, 3, 4, 5, 6, 7, or more) acidic residues (D and/or E) in sequence. In some embodiments, the acidic peptide segment comprises up to 7 residues in length and comprises at least 50% acidic residues. In some embodiments, an acidic peptide segment comprises (Xa) 1-7, wherein each Xa is independently D or E. In some embodiments, an acidic peptide segment comprises EE or EEE.

In some embodiments, peptide amphiphiles comprise a basic peptide segment. For example, in some embodiments, the acidic peptide comprises one or more (e.g., 1, 2, 3, 4, 5, 6, 7, or more) basic residues (R, H, and/or K) in sequence. In some embodiments, the basic peptide segment comprises up to 7 residues in length and comprises at least 50% basic residues. In some embodiments, an acidic peptide segment comprises (Xb).sub.1-7, wherein each Xb is independently R. H, and/or K.

In some embodiments, peptide amphiphiles comprises a structural and/or beta-sheet-forming segment. In some embodiments, the structural segment is rich in H, I, L, F. V, and A residues. In some embodiments, the structural and/or beta-sheet-forming segment comprises an alanine- and valine-rich peptide segment (e.g., AAVV (SEQ ID NO: 2), AAAVVV (SEQ ID NO: 3) VVAA (SEQ ID NO: 4). VVVAAA (SEQ ID NO: 5), or other combinations of V and A residues, etc.). In some embodiments, the structural and/or beta sheet peptide comprises 4 or more consecutive A and/or V residues, or conservative or semi-conservative substitutions thereto. In some embodiments, the structural and/or beta-sheet forming peptide segment comprises 4 or more consecutive non-polar aliphatic residues (e.g., alanine (A), valine (V), leucine (L), isoleucine (I), methionine (M)). In some embodiments, the structural and/or beta-sheet forming peptide segment comprises 2-16 amino acids in length and comprises 4 or more (e.g., 2, 3, 4, 5, 6, 7, 8, 9, 10, 11, 12, 13, 14, 15, 16, or ranges there between) non-polar aliphatic residues.

In some embodiments, peptide amphiphiles comprise a non-peptide spacer or linker segment. In some embodiments, the non-peptide spacer or linker segment is located at the opposite terminus of the peptide from the hydrophobic segment. In some embodiments, the spacer or linker segment provides the attachment site for a bioactive group. In some embodiments, the spacer or linker segment provides a reactive group (e.g., alkene, alkyne, azide, thiol, maleimide etc.) for functionalization of the PA. In some embodiments, the spacer or linker is a substantially linear chain of CH₂, O, (CH₂)₂O, O(CH₂)₂, NH, and C═O groups (e.g., CH₂(O(CH₂)₂)₂NH, CH₂(O(CH₂)₂)₂NHCO(CH₂)₂CCH, etc.). In some embodiments, a spacer or linker further comprises additional bioactive groups, substituents, branches, etc.

Suitable peptide amphiphiles, PA segments, PA nanostructures, and associated reagents and methods are described, for example in U.S. Pat. Nos. 11,066,444; 10,792,327; 10,752,656; 10,738,294; 10,689,252; 10,316,432; 10,316,180; 9,926,195; 9,650,421; 9,556,232; 9,517,275; 9,512,404; 9,169,294; 8,940,858; 8,834,840; 8,772,228; 8,748,569; 8,850,923; 8,512,693; 8,450,271; 8,138,140; 8,124,583; 8,114,835; 8,114,834; 8,080,262; 8,063,014; 7,851,445; 7,838,491; 7,745,708; 7,683,025; 7,554,021; 7,544,661; 7,534,761; 7,491,690; 7,452,679; 7,390,526; 7,371,719; 6,890,654; herein incorporated by reference in their entireties.

The characteristics (e.g., shape, rigidity, hydrophilicity, etc.) of a PA supramolecular structure depend upon the identity of the components of a peptide amphiphile (e.g., lipophilic segment, acidic segment, structural segment, bioactive segment, etc.). For example, nanofibers, nanospheres, intermediate shapes, and other supramolecular structures are achieved by adjusting the identity of the PA component parts. In some embodiments, characteristics of supramolecular nanostructures of PAs are altered by post-assembly manipulation (e.g., heating/cooling, stretching, etc.).

In some embodiments, a peptide amphiphile comprises: (a) a hydrophobic tail comprising an alkyl chain of 8-24 carbons; (b) a structural segment comprising or consisting of 4-8 V and A residues; and (c) a charged segment (e.g., comprising EE, EEE, etc.). In some embodiments, any PAs within the scope described herein, comprising the components described herein, or within the skill of one in the field, may find use.

In some embodiments, peptide amphiphiles comprise a bioactive moiety. In particular embodiments, a bioactive moiety is the C-terminal or N-terminal most segment of the PA (e.g., opposite end from the hydrophobic tail). In some embodiments, the bioactive moiety is attached to the end of the charged segment. In some embodiments, the bioactive moiety is exposed on the surface of an assembled PA structure (e.g., nanofiber). A bioactive moiety is typically a peptide, but is not limited thereto. Examples described in detail herein utilize a peptide sequence that binds BMP-2 as a bioactive moiety. In some embodiments, a bioactive peptide is a therapeutic peptide. Bioactive peptides and other moieties for achieving functionality will be understood. In some embodiments, bioactive moieties are provided having binding affinity for a target protein of less than 10 μM, less than 100 μM, less than 1 μM, less than 100 nM, less than 10 nM, less than 1 nM, etc.

In some embodiments, the bioactive peptide is a binding peptide capable of binding to a bioactive factor (e.g., protein or other bioactive molecule) that is relevant to the intended purpose of the material. In some embodiments, binding of the bioactive peptide to the bioactive factor results in the bioactive factor being associated with the PA nanofiber.

In some embodiments, the bioactive peptide is a peptide mimetic of a bioactive factor (e.g., protein or other bioactive molecule) that is relevant to the intended purpose of the material. In such embodiments, the bioactive peptide mimics the function of the bioactive factor, rather than recruiting the bioactive factor to the nanofiber.

In some embodiments, PAs herein display a bioactive peptide capable of binding to or mimicking a function of a bioactive factor selected from protein/polypeptide agents, such as an enzyme, a receptor, a channel protein, a hormone, a cytokine, a growth factor, and antibody drug. In some embodiments, the bioactive factor finds use in the repair of tissue/bone defects and/or generation/regeneration of tissue/bone. Suitable bioactive factors include bone morphogenic proteins (e.g., BMP-1, BMP-2, BMP-4, BMP-6, and BMP-7); members of the transforming growth factor beta (TGF-β) superfamily including, but not limited to, TGF-β1, TGF-β2, and TGF-β3; epidermal growth factor (EGF), transforming growth factor-alpha (TGF-α), growth differentiation factors (GDF1, GDF2, GDF3. GDF5, GDF6, GDF7, myostatin/GDF8, GDF9, GDF10, GDF11, and GDF15); human endothelial cell growth factor (ECGF); granulocyte macrophage colony stimulating factor (GM-CSF); nerve growth factor (NGF); vascular endothelial growth factor (VEGF); fibroblast growth factor (FGF); insulin-like growth factor (IGF); cartilage derived morphogenetic protein (CDMP): platelet rich plasma (PRP); platelet derived growth factor (PDGF); insulin growth factor one (IGF-I); or any combinations thereof.

In a particular embodiment exemplified herein, the bioactive factor is BMP-2, and the PA nanofibers of the composite material display a BMP-2 binding peptide. In some embodiments, the BMP-2 binding peptide is of the sequence TSPHVPYGGGS (SEQ ID NO: 1). In some embodiments, other bioactive peptides capable of mimicking and/or binding to bioactive factors are understood and have been demonstrated to be useful when displayed on PA nanofibers. Embodiments herein are not limited to use with a BMP-2 binding peptide. In some embodiments, a bioactive peptide binds BMP-2 and has at least 70% (e.g., 70%, 75%, 80%, 85%, 90%, 95%, 100%, or more, or ranges therebetween) sequence identity with one of SEQ ID NO: 1 (TSPHVPYGGGS). In some embodiments, a bioactive peptide binds BMP-2 and has at least 70% (e.g., 70%, 75%, 80%, 85%, 90%, 95%, 100%, or more, or ranges therebetween) sequence similarity (e.g., conservative or semi-conservative) with SEQ ID NO: 1 (TSPHVPYGGGS). In some embodiments, a bioactive peptide binds BMP-2 and has 4 or fewer (e.g., 4, <4, 3, <3, 2, <2, 1, 0) substitutions relative to SEQ ID NO: 1 (TSPHVPYGGGS). In some embodiments, a bioactive peptide binds BMP-2 and has 4 or fewer (e.g., 4, <4, 3, <3, 2, <2, 1, 0) non-conservative substitutions relative to SEQ ID NO: 1 (TSPHVPYGGGS). In some embodiments, a bioactive peptide binds BMP-2 and has 4 or fewer (e.g., 4, <4, 3, <3, 2, <2, 1, 0) semi-conservative substitutions relative to SEQ ID NO: 1. In some embodiments, a bioactive peptide binds BMP-2 and has 4 or fewer (e.g., 4, <4, 3, <3, 2, <2, 1, 0) conservative substitutions relative to SEQ ID NO: 1 (TSPHVPYGGGS).

In some embodiments, a bioactive peptide amphiphile comprises: (a) a hydrophobic tail comprising an alkyl chain of 8-24 carbons; (b) an alanine- and valine-rich peptide segment (e.g., AAVV (SEQ ID NO: 2), AAAVVV (SEQ ID NO: 3) VVAA (SEQ ID NO: 4), VVVAAA (SEQ ID NO: 5), or other combinations of V and A residues, etc.); (c) a charged segment (e.g., comprising EE or EEE, etc.), and (d) a bioactive peptide (e.g., BMP-2 binding peptide (e.g., SEQ ID NO: 1 (TSPHVPYGGGS))).

In some embodiments, a diluent peptide amphiphile comprises: (a) a hydrophobic tail comprising an alkyl chain of 8-24 carbons; (b) an alanine- and valine-rich peptide segment (e.g., AAVV (SEQ ID NO: 2), AAAVVV (SEQ ID NO: 3) VVAA (SEQ ID NO: 4), VVVAAA (SEQ ID NO: 5), or other combinations of V and A residues, etc.); (c) a charged segment (e.g., comprising EE or EEE, etc.),

In some embodiments, a bioactive or diluent PA further comprises an attachment segment or residue (e.g., K) for attachment of the hydrophobic tail to the peptide portion of the PA. In some embodiments, the hydrophobic tail is attached to a lysine side chain. In other embodiments a hydrophobic tail is attached directly to the terminal amino acid of the structural peptide segment.

In some embodiments, a bioactive peptide amphiphile comprises (e.g., from C-terminus to N-terminus or from N-terminus to C-terminus): bioactive peptide (e.g., BMP-2 binding peptide)—charged segment (e.g., comprising EE, EEE, etc.)—structural segment (e.g., comprising VVAA (SEQ ID NO: 4), AAVV (SEQ ID NO: 2), VA, AV, etc.)—hydrophobic tail (e.g., comprising an alkyl chain of 8-24 carbons).

In some embodiments, a bioactive peptide amphiphile comprises (e.g., from C-terminus to N-terminus or from N-terminus to C-terminus): bioactive peptide (e.g., BMP-2 binding peptide)—charged segment (e.g., comprising EE, EEE, etc.)—structural segment (e.g., comprising VVAA (SEQ ID NO: 4), AAVV (SEQ ID NO: 2), VA, AV, etc.)—attachment segment or peptide (e.g., K)—hydrophobic tail (e.g., comprising an alkyl chain of 8-24 carbons).

In some embodiments, a bioactive peptide amphiphile comprises (e.g., from C-terminus to N-terminus or from N-terminus to C-terminus): TSPHVPYGGSEEEAAVVVK-C₁₂ (SEQ ID NO: 6).

In some embodiments, a diluent peptide amphiphile comprises (e.g., from C-terminus to N-terminus or from N-terminus to C-terminus): charged segment (e.g., comprising EE, EEE, etc.)—structural segment (e.g., comprising VVAA (SEQ ID NO: 4), AAVV (SEQ ID NO: 6), VA, AV, etc.)—hydrophobic tail (e.g., comprising an alkyl chain of 8-24 carbons).

In some embodiments, a diluent peptide amphiphile comprises (e.g., from C-terminus to N-terminus or from N-terminus to C-terminus): charged segment (e.g., comprising EE, EEE, etc.)—structural segment (e.g., comprising VVAA (SEQ ID NO: 4), AAVV (SEQ ID NO: 6), VA, AV, etc.)—attachment segment or peptide (e.g., K)—hydrophobic tail (e.g., comprising an alkyl chain of 8-24 carbons).

In some embodiments, a diluent peptide amphiphile comprises (e.g., from C-terminus to N-terminus or from N-terminus to C-terminus): EEEAAAVVV—C₁₆ (SEQ ID NO: 7).

In some embodiments, provided herein are nanofibers and nanostructures assembled from the peptide amphiphiles described herein. In some embodiments, a nanofiber is prepared by the self-assembly of the PAs described herein. In some embodiments, a nanofiber comprises or consists of PAs displaying a bioactive peptide. In some embodiments, the bioactive peptides are displayed on the surface of the nanofiber. In some embodiments, in addition to PAs displaying biactive peptides, diluent PAs are included in the nanofibers. In some embodiments, diluent PAS are peptide amphiphiles, as described herein (e.g., structural segment, charged segment, hydrophobic segment, etc.), but lacking a bioactive peptide. In some embodiments, the diluent PAs and bioactive PAs self-assemble into a nanofiber comprising both types of PAs. In some embodiments, nanostructures (e.g., nanofibers) assembled from the peptide amphiphiles described herein are provided.

In some embodiments, nanostructures are assembled from (1) PAs bearing a bioactive moiety (e.g., BMP-2-binding moiety) and (2) filler PAs (e.g., PAs not-labeled or not displaying a bioactive moiety, etc.). In some embodiments, nanostructures (e.g., nanofibers) comprise: (i) less than 50% (e.g., 49%, 45%, 40%, 35%, 30%, 25%, 20%, 15%, 10%, 5%, 4%, 3%, 2%, 1%, or any ranges there between) PAs bearing a bioactive moiety (e.g., BMP-2-binding moiety). In some embodiments, nanostructures (e.g., nanofibers) comprise and at least 2% (e.g., 2%, 3%, 4%, 5%, 10%, 15%, 20%, 25%, 30%, 35%, 40%, 45%, 50%, 55%, 60%, 65%, 70%, 75%, 80%, 85%, 90%, 95%, 98%, or any ranges there between) PAs bearing a bioactive moiety (e.g., BMP-2-binding moiety). In some embodiments, nanofibers comprise at least 50% (e.g., 50%, 55%, 60%, 65%, 70%, 75%, 80%, 85%, 90%, 95%, 98%, or any ranges there between) filler peptide amphiphiles.

In some embodiments, the ratio of PAs bearing a bioactive moiety to filler PAS determines the density of bioactive moieties (e.g., BMP-2-binding moiety) displayed on the nanostructure surface.

In some embodiments in which a PA or PA nanofiber is provided that is capable of binding to a bioactive factor (e.g., nanofiber displaying a BMP-2 binding peptide), the PA or PA nanofiber is provided in a composition (e.g., composite material) comprising such a bioactive factor. As discussed above, bioactive factors that may find use in compositions herein (e.g., with bioactive PAs capable of binding such factors) include bone morphogenic proteins (e.g., BMP-1, BMP-2, BMP-4, BMP-6, and BMP-7); members of the transforming growth factor beta (TGF-β) superfamily including, but not limited to, TGF-β1, TGF-β2, and TGF-β3; epidermal growth factor (EGF), transforming growth factor-alpha (TGF-α), growth differentiation factors (GDF1, GDF2, GDF3. GDF5, GDF6, GDF7, myostatin/GDF8, GDF9, GDF10, GDF11, and GDF15); human endothelial cell growth factor (ECGF); granulocyte macrophage colony stimulating factor (GM-CSF); nerve growth factor (NGF); vascular endothelial growth factor (VEGF); fibroblast growth factor (FGF); insulin-like growth factor (IGF); cartilage derived morphogenetic protein (CDMP); platelet rich plasma (PRP); insulin growth factor one (IGF-I); platelet-derived growth factor (PDGF); or any combinations thereof. Embodiments herein are not limited to only these bioactive factors.

In other embodiments in which a PA or PA nanofiber is provided that is capable of binding to a bioactive factor (e.g., nanofiber displaying a BMP-2 binding peptide), the PA or PA nanofiber is provided in a composition (e.g., composite material) lacking exogenous bioactive factor. In some embodiments, a PA or PA nanofiber is provided that is capable of binding to endogenous bioactive factor and exerting a therapeutic benefit, and is therefore provided without exogenous bioactive factor.

In some embodiments, compositions herein (e.g., composite materials) comprise one or more polymer materials. In some embodiments, the polymer is provided to allow the material to be molded into a desired shape. In some embodiments, the polymer provides structural integrity to the compositions. Suitable polymers for use in certain embodiments herein may include hydroxypropyl cellulose, hydroxypropyl methylcellulose, hydroxyethyl methylcellulose, carboxymethylcellulose, hydroxyethylcellulose and salts thereof, Carbopol, poly(hydroxyethylmethacrylate), poly(methoxyethylmethacrylate), poly(methoxyethoxy-ethylmethacrylate), polymethylmethacrylate (PMMA), methylmethacrylate (MMA), gelatin, polyvinyl alcohols, propylene glycol, PEG 200, PEG 300, PEG 400, PEG 500, PEG 550, PEG 600, PEG 700, PEG 800, PEG 900, PEG 1000, PEG 1450, PEG 3350, PEG 4500, PEG 8000 or combinations thereof.

In particular embodiments, compositions herein (e.g., composite materials) comprise one or more polyethylene glycols (e.g., PEG 1450, PEG 600, etc.). In some embodiments, compositions herein (e.g., composite materials) comprise PEG 1450 and PEG 600.

In some embodiments, the polymer component(s) comprises up to 95 wt % (e.g., 95%, 90%, 85%, 80%, 75%, 70%, 65%, 60%, 55%, 50%, 40%, 30%, or ranges therebetween) of the components of the composition.

In some embodiments, compositions herein (e.g., composite materials) comprise one or more ceramic materials. In some embodiments, ceramic component(s) provide compression resistance to the material. Suitable ceramics include hydroxyapatite (HA), tricalcium phosphate (TCP), bioglass, calcium sulfate, etc. In some embodiments, compositions comprise HA and/or TCP. In some embodiments, the ceramic is provided as a particulate. In some embodiments, the ceramic is homogenously disposed throughout the material. In some embodiments, a ceramic (e.g., HA, TCP) has a particle diameter (e.g., mean diameter of particles in a population) of 10-2000 μm (e.g., 10 μm, 20 μm, 30 μm. 40 μm, 50 μm, 60 μm, 70 μm, 80 μm, 90 μm, 100 μm, 200 μm, 300 μm, 400 μm, 500 μm, 600 μm, 700 μm, 800 μm, 900 μm, 1000 μm, 1500 μm, 2000 μm, or ranges therebetween).

In some embodiments, the ceramic component(s) comprises 5-50 wt % (e.g., 5%, 10%, 25%, 30%, 35%, 40%, 45%, 50%, or ranges therebetween) of the components of the composition.

Provided herein are compositions comprising composite materials, particularly for medical uses. In some embodiments, the composite materials comprise peptide amphiphile nanostructures, a polymer component, and a ceramic component.

In some embodiments, a composite material comprises 5 wt % or less (e.g., 5%, 4%, 3%, 2%, 1%, 0.5%, 0.2%, 0.1%, or less, or ranges therebetween) peptide amphiphile nanofibers. In some embodiments, a composite material comprises 50 wt % and 90 wt % (e.g., 50%, 55%, 60%, 65%, 66%, 67% 68%, 69%, 70% 71%, 72%, 73%, 74%, 75%, 80%, 85%, 90%, or ranges therebetween) polymer component(s) (e.g., PEG 1450 and PEG600 combined). In some embodiments, a composite material comprises 35 wt % and 55 wt % (e.g., 35%, 40%, 41%, 42%, 43%, 44%, 45%, 46%, 47%, 48%, 49%, 50%, 55%, or ranges therebetween) of a first polymer component (e.g., PEG 1450). In some embodiments, a composite material comprises 15 wt % and 35 wt % (e.g., 15%, 20%, 21%, 22%, 23%, 24%, 25%, 26%, 27%, 28%, 29%, 30%, 35%, or ranges therebetween) of a second polymer component (e.g., PEG 600). In some embodiments, a composite material comprises 15 wt % and 45 wt % (e.g., 15%, 20%, 25%, 26%, 27% 28%, 29%, 30% 31%, 32%, 33%, 34%, 35%, 40%, 45%, or ranges therebetween) ceramic component(s) (e.g., HA or TCP). In some embodiments, a composite material comprises 5 wt % or less (e.g., 5%, 4%, 3%, 2%, 1%, 0.5%, 0.2%, 0.1%, or less, or ranges therebetween) bioactive factor (e.g., BMP-2).

An exemplary composite material comprises <1 wt % diluent PA, <1 w % bioactive PA, 46 wt % PEG 1450, 23% PEG 600, 30 wt % TCP, and <1 wt % BMP-2.

In some embodiments, the composite materials are devoid of water (e.g., <0.1 wt %, <0.05 wt %, <0.02 wt %, <0.01 wt %, <0.005 wt %, <0.002 wt %, <0.001 wt %, etc.). In some embodiments, the composite materials and/or one or more components thereof have been treated (e.g., freeze dried, lyophilized, etc.) to remove water from the composite and/or component. In some embodiments, the bioactive components (e.g., bioactive peptide, bioactive factor, etc.) are active when delivered within a dry composite material.

In some embodiments, materials herein allow for lower therapeutic BMP-2 dose for successful spina fusion by orders of magnitude (e.g., a 10×lower dose of rhBMP-2 than water-based PA, 1000× reduction compared to the clinical dose).

The compositions described herein find use in medical and veterinary applications, such as bone/tissue repair, delivery of bioactive factors, regenerative medicine, etc. A exemplary composite material described herein finds use for bone regeneration (e.g., for spinal fusion applications). However, composites can be made using the technology described herein that comprise peptide amphiphile nanofibers displaying different bioactive peptides and/or contain different bioactive factors. For example, bioactive peptides and/or factors included in the composites can be tailored to promote cell adhesion or mimic the function of growth factors. In addition to bone repair, materials may find use in the regeneration/repair of cartilage or other connective tissue, muscle, skin, etc. In some embodiments, the relative percentages and types of polymer and/or ceramic can be adjusted to create composites that are more or less compressible, moldable, viscous, stable, biodegradable, etc.

In some embodiments, methods are provided for administering a composite material described herein to a subject. In the exemplary case of a bone defect or injury (e.g., to create a spinal fusion), a material described herein is molded into the treatment site. In some embodiments, the exemplary materials described herein promote formation of bone at the treatment site.

In some embodiments, material described herein find use in posterolateral spinal fusion surgeries, where bone graft substitutes are implanted into the spinal muscular space. The treatment site has irregular geometry, requiring a moldable material. Furthermore, once the surgical site is closed, the surrounding muscular tissue will apply force onto the implant and movement of the patient can also displace it. The implant must maintain its integrity, stay in place, and resist compression against these forces. The exemplary materials herein find use in meeting the requirement of this procedure.

### EXPERIMENTAL

### Example 1

### Composite Synthesis

Provided below is a procedure for synthesis of an exemplary composite material within the scope herein. The composite material described below finds particular use in the repair of bone defects. Alternative components, procedures, materials, and uses are contemplated and within the scope herein.

Step 1a: Prepare Peptide Amphiphile Solutions Containing rhBMP-2

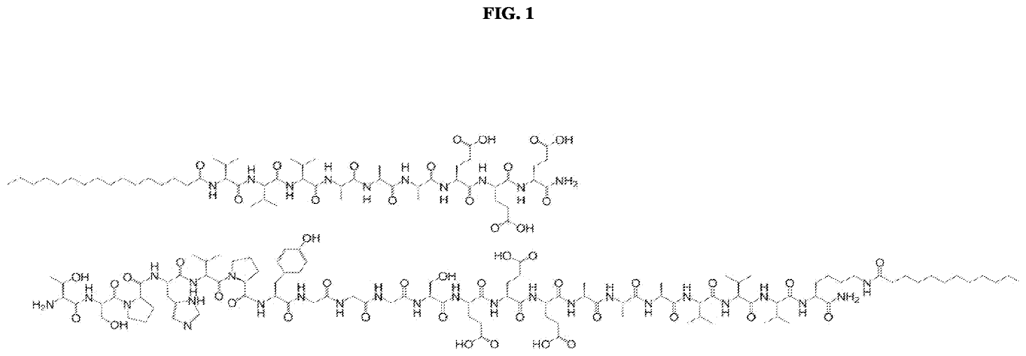

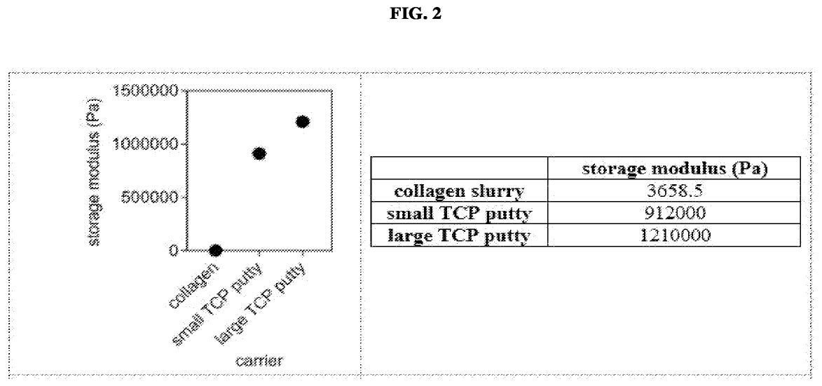

A bioactive PA supramolecular polymer has been identified that can potentiate the signal of rhBMP-2, thus reducing the therapeutic dose necessary to achieve bone growth (Lee, S. S., Hsu, E. L., Mendoza, M., Ghodasra, J., Nickoli, M. S., Ashtekar, A., Polavarapu, M., Babu, J., Riaz, R. M., Nicolas, J. D. and Nelson, D., 2015. Gel scaffolds of BMP-2-binding peptide amphiphile nanofibers for spinal arthrodesis. Advanced healthcare materials, 4(1), pp. 131-141.; incorporated by reference in its entirety). This bioactive PA finds use in bone repair applications such as spinal fusion surgeries, because existing rhBMP-2 products require supraphysiological doses to achieve sufficient bone growth, which (while effective) can lead to dangerous side effects. This PA-based supramolecular polymer contains two PA molecules, a bioactive PA capable of binding BMP-2 and a diluent PA with no bioactive component (FIG. 1). The bioactive PA supramolecular polymer was prepared by dissolving to 1.2 wt % (12 mg/mL) of diluent PA in water with 15 mM NaOH, dissolving to 1.2 wt % (12 mg/mL) bioactive PA in water with 30 mM NaOH, mixing equal volumes of the PA solutions, so the final PA solution has equal wt % of both PAs (0.6 wt % each), and heating (thermally annealing) the PA mixture at 80 degrees Celsius (e.g., in a water bath or oven) for 30 minutes, and then allowing the mixture to cool to room temperature. Generally, the PA solution is allowed to passively cool slowly overnight, but shorter or longer cooling times may be used. An rhBMP-2 solution is added to the cooled to PA mixture to a final PA concentration of 1 wt % (10 mg/mL).

The rhBMP-2 concentration can be adjusted to desired amount. As an example, when this supramolecular polymer was tested in a rat spinal fusion model, the desired rhBMP-2 dose was 10-100 ng/animal (5-50 ng/scaffold, two scaffolds per animal). At the size scale of the rat spine, each scaffold requires around 130 uL of PA solution, delivered through a structural component such as the polymer/ceramic putty. Thus, a solution that contains 1 wt % PA and 50 ng rhBMP-2 in 130 uL would be desired and prepared as follows: begin with a rhBMP-2 solution that is 2.307 ug/mL (2307 ng/mL), add 50 uL of rhBMP-2 solution to 250 uL of 1.2 wt % annealed PA solution. This results in a solution that is 1 wt % PA and 384.5 ng/mL rhBMP-2, or 0.3845 ng/uL rhBMP-2. Since the solution contains 0.3845 ng/uL rhBMP-2, 130 uL of the solution will contain 50 ng of rhBMP-2.

For the particular purpose described in Step 1a (creating PA implants for a rat spinal fusion model), exemplary solutions containing 1 wt % PA and 0.3845 ng/uL rhBMP-2 have been identified as favorable. For other embodiments of this invention, the supramolecular polymer may be at a different concentration in solution, or the solution may contain a different concentration or type of bioactive growth factor or other therapeutic agent. For example, the PA solution may be mixed with recombinant transforming growth factor beta-1 (TGFβ-1). In addition to growth factors and peptide mimetics thereof, the PA solution may also be mixed with a small molecule drug such as resveratrol. Furthermore, the PA solution may contain no exogenous growth factor or therapeutic agent at all; for example, if the supramolecular polymer is used to bind endogenous growth factors once implanted in the body.

### Step 1b: Prepare Polymer/Ceramic Putty

The bioactive component prepared in Step 1a (supramolecular polymer and rhBMP-2) is a liquid solution that cannot be implanted directly into a defect. The implant must be able to maintain its shape and stay in place, and surgeons would ideally be able to mold it to fit irregular geometries. A composite putty of the bioactive component is made by weighing out a ratio of 46.7% PEG 1450, 23.3% PEG 600, ceramic 30% (PEG: polyethylene glycol polymer). In this exemplary embodiment of the invention for spinal fusion, the ceramic is tri-calcium phosphate (TCP) particles. As an example, if a final 700 mg of putty is desired, the following materials would be weighed out: 326.9 mg PEG 1450, 163.1 mg PEG 600, 210 mg TCP. This particular combination of polymers has been found to result in a putty-like material with favorable surgical handling properties at room temperature; however, any water-soluble material with these handling properties may be used in embodiments within the scope herein. For example, other polymers that could be used include polyethylene glycol, polyacrylic acid copolymer, and polyvinyl alcohol.

The two PEG components were placed in the same container, and heated at 50° C. until a clear liquid is observed. At the example amounts described above, if 50° C. is used for heating, approximately 15 minutes of heating was sufficient. After a clear liquid was observed, the PEG solution was removed from heat. The PEG polymers will start to form a white, waxy solid almost immediately. The TCP particles were added this waxy solid.

Step 2: Combine PA Solution (Containing any Added Growth Factors or Pharmaceutical Agents) with Putty

The PA solution was added to the PEG and TCP mixture. For each 700 mg of putty, 520 uL of PA solution was added. The water from the PA solution immediately dissolves the PEG into a “slush-like” material. The material was mixed well to evenly distribute the TCP particles. The mixture of PEG, TCP, PA, and rhBMP-2 was freeze-dried to remove water. The resultant material was compressed and shaped into a putty material. The PEG provides moldability and the TCP provides compression resistance. In the exemplary embodiment of the invention for spinal fusion, the PA and rhBMP-2 are bioactive components contained within the putty. Other drying methods may be used to remove water from the material, such as evaporation.

### Example 2

### Effect of TCP on Handling Properties and Biological Function

TCP comes in many different forms. For the exemplary embodiment of the invention for spinal fusion described in Example 1, “small” or “large” TCP particles were investigated. The small TCP particles were 53-250 μm in size, while the large TCP particles were 250-1000 μm in size. The small TCP particles handle like a powder, while the large TCP particles are visually distinct particulates. Consequently, the “small TCP putty” was a smooth putty where individual particles are not detectable during handling, while the “large TCP putty” feels rougher during handling (particles detectable by touch). Addition of small or large TCP results in a putty with improved handling properties. The putty material holds together, has sufficient compression resistance for implantation into defects, and exhibits an increased storage moduli (over 200×) compared to material lacking TCP. Furthermore, surgeons experienced in spinal fusion surgeries tested the putties, and determined that the large TCP particle version had more compression resistance, although both had acceptable handling properties for direct implantation into irregularly shaped defects. This is because large TCP particles are superior at resisting compression compared to small TCP particles that handle like a powder, and is reflected in the slightly higher storage modulus of the large TCP putty.

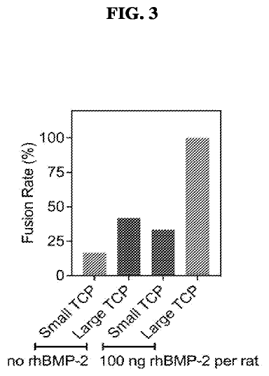

The size of the TCP particles (small: 53-250 μm, large: 250-1000 μm) affects the bioactivity of the putty. In the embodiment of the invention for spinal fusion, when the putty is made with large TCP particles, the material can achieve 100% fusion in a rat spinal fusion model with 100 ng rhBMP-2 per rat (FIG. 3). In this animal model, this is considered a low-dose of rhBMP-2, which is advantageous because supraphysiological rhBMP-2 doses can lead to undesired and dangerous side effects. When the large TCP particles are replaced with an equal weight of small TCP particles, and all other putty components are the same, the fusion rate is significantly lower (FIG. 3). Even when the putties contain no BMP-2, some fusion was observed (FIG. 3). Large TCP putty achieves a fusion rate of around 42% when delivering no BMP-2 (FIG. 3), indicating some recruitment of endogenous growth factor. Small TCP putty delivering no BMP-2 achieves less fusion compared to large TCP putty (17% vs. 42%), indicating that the TCP particles provide a degree of bioactivity.

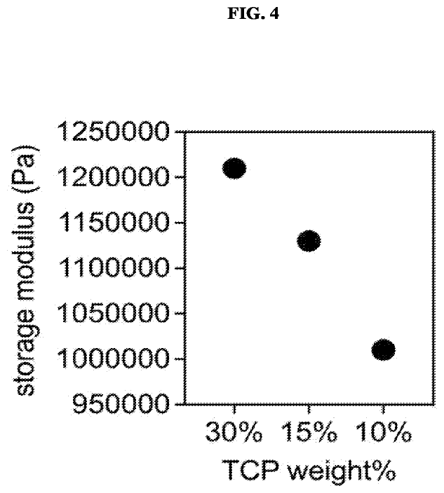

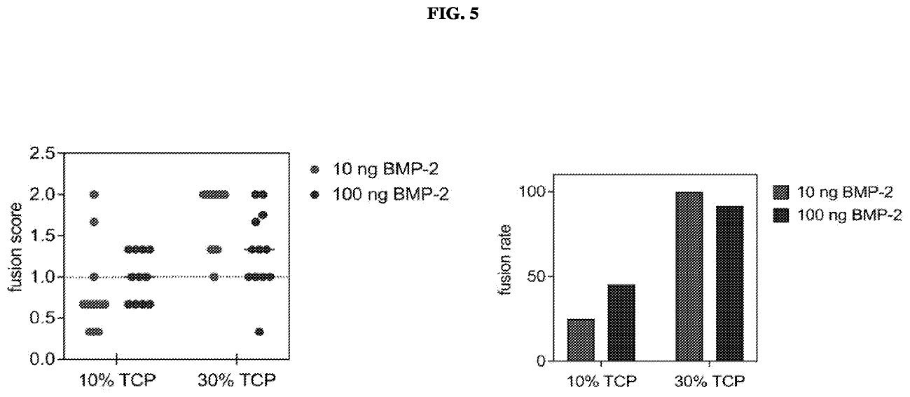

In the PEG/TCP carrier for the bioactive supramolecular polymer and rhBMP-2, the ratio of PEG to TCP may affect mechanical and handling properties. PEG is water soluble while TCP is not, which can affect in-vivo implant degradation rate and also bioactivity. An exemplary embodiment containing 30% large TCP particles by weight was able to achieve 100% in a rat spinal fusion model with a low dose of rhBMP-2 (FIG. 3). In addition to favorable biological function, this embodiment also had favorable handling properties as verified by surgeons and shown with rheological experiments. Different ratios of TCP particles were explored by varying the TCP wt % to 30%, 15%, and 10%. When these putties were examined by surgeons experienced in spinal fusion surgeries, putties with 15% and 10% TCP by weight were found to be less compression-resistant than putties with 30% TCP by weight. However, all were deemed usable for spinal fusion implantation. Lower TCP percentages correlated with slightly lower storage moduli of the putties (FIG. 4).

The TCP weight % in the putty can also affect bioactivity. When TCP comprises 30% of the putty by weight, the putty is more effective at promoting spinal fusion at low BMP-2 dosages in a rat model, even achieving 100% fusion at 10 ng rhBMP-2 (FIG. 5). For the purpose of inducing bone growth to promote spinal fusion, 30% TCP by weight appears to be highly favorable, resulting in the unprecedented 1000-fold dose reduction compared to the therapeutic dose used clinically. For other therapeutic applications, other ratios of TCP relative to the other components may be desired.

### Example 3

### Further Characterization and Testing

PA Structures within Putty

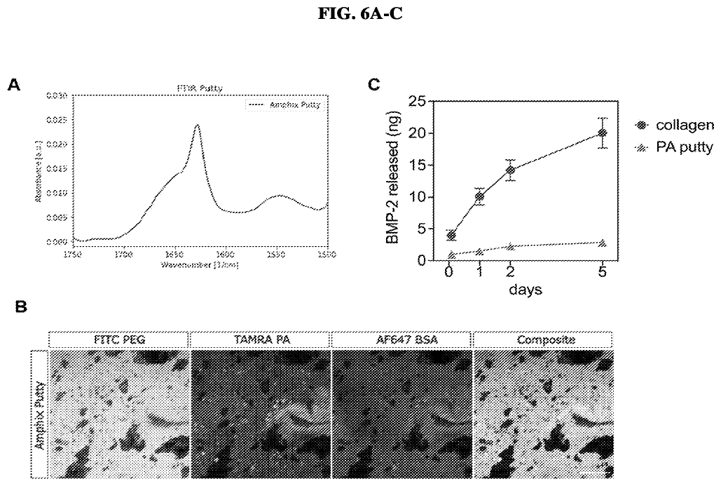

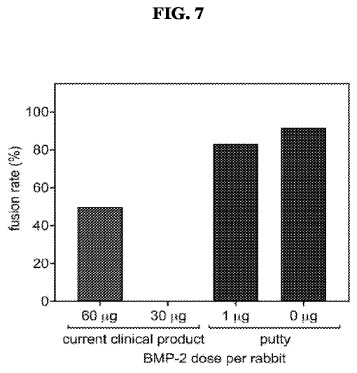

The putty with 30% large TCP particles was further characterized. In some embodiments, in aqueous solution, PA nanofibers comprise PA molecules held together by non-covalent β-sheeted structures. In the putty, the PA does not exist in aqueous solution but instead among, for example, PEG polymer chains. FTIR (Fourier transform infrared spectroscopy) experiments on an exemplary putty show the presence of β-sheeted structures (FIG. 6A), indicating that the PA is still assembled into nanofibrous structures within the non-aqueous putty.

### Protein Distribution Within Putty

The BMP-2 binding PA contains an epitope designed to bind BMP-2 growth factor; however, the negatively charged nanofibers can nonspecifically bind charged proteins. To visualize protein distribution within the putty, fluorescently-tagged bovine serum albumin (BSA, considered a “model” protein) was loaded into a putty containing fluorescently-tagged PA as well as fluorescently-tagged PEG (FIG. 6B). These images show the colocalization of PA with BSA (FIG. 6B).

BMP-2 Release from Putty with PA

One limitation of existing clinical products, that use collagen carriers to deliver BMP-2, is that collagen has no affinity for BMP-2. Thus, burst release of BMP-2 occurs once implanted, so the BMP-2 does not remain at the site where new bone is needed. The PA nanofibers are designed to bind BMP-2 growth factor and retain it at the implant site. When BMP-2 is loaded into both collagen and putty materials, the putty shows a slower release rate (FIG. 6C).

### Large Animal Model

Following success in a small animal model (rat spinal fusion), putty with 30% large TCP was tested in a widely accepted stringent large animal model (rabbit posterolateral spinal fusion). For comparison, ACS (absorbable collagen sponge, used in INFUSE™, a current clinical product) implants delivering BMP-2 were also tested. ACS implants delivering 60 ug BMP-2 per rabbit achieved a 50% fusion rate, and when the BMP-2 dose was reduced to 30 μg, the fusion rate was 0% (FIG. 7). However, when the putty was used to deliver an ultra-low dose of 1 μg BMP-2 per rabbit, around 83% of animals successfully fused (FIG. 7). A putty containing no BMP-2 achieved around 92% fusion (FIG. 7), indicating recruitment of endogenous BMP-2 and bioactivity of the TCP particles. In the rat model, the putty containing no BMP-2 achieved a 42% fusion rate, lower than the 92% achieved in rabbits, a larger animal with a larger defect size. This is likely due to variation in bone decortication procedures during surgery, since the amount of decortication to expose bone progenitor cells can affect the capacity for bone regeneration. The data indicate that the putty with no BMP-2 has capacity to induce bone growth with no osteogenic growth factor.

SEQUENCES

| | |
|---|---|
| SEQ ID NO: 1 | TSPHVPYGGGS |
| SEQ ID NO: 2 | AAVV |
| SEQ ID NO: 3 | AAAVVV |
| SEQ ID NO: 4 | VVAA |
| SEQ ID NO: 5 | VVVAAA |
| SEQ ID NO: 6 | TSPHVPYGGSEEEAAVVVK |
| SEQ ID NO: 7 | EEEAAAVVV |

1. A composition comprising a composite material comprising:
(a) peptide amphiphile nanofibers;
(b) a polymer component; and
(c) a ceramic component.

2. The composition of claim 1, wherein the peptide amphiphile nanofibers comprise a supramolecular assembly of peptide amphiphiles, the peptide amphiphiles comprising:
(i) a hydrophobic non-peptidic segment;
(ii) a structural peptide segment; and
(iii) a charged segment.

3. The composition of claim 1, wherein the polymer component comprises polyethylene glycol (PEG).

4. The composition of claim 3, wherein the polymer component comprises a PEG selected from PEG 200, PEG 300, PEG 400, PEG 500, PEG 550, PEG 600, PEG 700, PEG 800, PEG 900, PEG 1000, PEG 1450, PEG 3350, PEG 4500, PEG 8000 or combinations thereof.

5. The composition of claim 4, wherein the polymer component comprises PEG 600 and PEG 1450.

6. The composition of claim 1, wherein the ceramic component comprises hydroxyapatite (HA), tricalcium phosphate (TCP), bioglass, or calcium sulfate.

7. The composition of claim 6, wherein the ceramic component comprises TCP.

8. The composition of claim 1, wherein the composite material further comprises a bioactive factor.

9. The composition of claim 1, wherein the composite material comprises <5 wt % of the peptide amphiphile nanofibers, 60-80 wt % of the polymer component, and 20-40 wt % of the ceramic component.

10. The composition of claim 9, wherein the composite material comprises <1 wt % of the peptide amphiphile nanofibers, 65-75 wt % of the polymer component, and 25-35 wt % of the ceramic component.

11. The composition of claim 10, wherein the composite material comprises <1 wt % of the peptide amphiphile nanofibers, about 69 wt % of the polymer component, and about 30 wt % of the ceramic component.

12. The composition of claim 2, wherein all or a portion of the peptide amphiphiles further comprise a bioactive peptide segment.

13. The composition of claim 12, wherein the bioactive peptide segment mimics the biological function of or is capable of binding to a bioactive factor and/or cellular component.

14. The composition of claim 13, wherein the bioactive factor and/or cellular component is selected from bone morphogenic proteins, transforming growth factors, epidermal growth factor, growth differentiation factors, human endothelial cell growth factor, granulocyte macrophage colony stimulating factor, nerve growth factor, vascular endothelial growth factor, fibroblast growth factor, insulin-like growth factor, cartilage derived morphogenetic protein, platelet rich plasma, platelet derived growth factor, insulin growth factor one, and platelet-derived growth factor.

15. The composition of claim 12, wherein the peptide amphiphile nanofibers comprise a supramolecular assembly of:
(i) bioactive peptide amphiphiles comprising (A) a hydrophobic non-peptidic segment, (B) a structural peptide segment, (C) a charged segment, and (D) a bioactive peptide; and
(ii) diluent peptide amphiphiles comprising (A) a hydrophobic non-peptidic segment, (B) a structural peptide segment, and (C) a charged segment.

16. The composition of claim 15, wherein (i) and (ii) are present at a ratio between 1:10 and 10:1.

17. The composition of claim 16, wherein (i) and (ii) are present are present at a ratio between 1:2 and 2:1.

18. The composition of claim 15, wherein the hydrophobic non-peptidic segment comprises an acyl chain.

19. The composition of claim 18, wherein the acyl chain comprises C₆-C₂₀.

20. The composition of claim 19, wherein the hydrophobic non-peptidic segment of the diluent peptide amphiphile and the hydrophobic non-peptidic segment of the bioactive peptide amphiphile the same length.

21. The composition of claim 19, wherein the hydrophobic non-peptidic segment of the diluent peptide amphiphile and the hydrophobic non-peptidic segment of the bioactive peptide amphiphile are different lengths.

22. The composition of claim 21, wherein the hydrophobic non-peptidic segment of the diluent peptide amphiphile is C₁₆ and the hydrophobic non-peptidic segment of the bioactive peptide amphiphile is C₁₂.

23. The composition of claim 15, wherein the structural peptide segment is an alanine- and valine-rich peptide segment.

24. The composition of claim 23, wherein the alanine- and valine-rich peptide segment comprises AAVV (SEQ ID NO: 2), AAAVVV (SEQ ID NO: 3) VVAA (SEQ ID NO: 4), or VVVAAA (SEQ ID NO: 5).

25. The composition of claim 15, wherein the charged peptide segment is a glutamate- and/or aspartate-rich segment.

26. The composition of claim 25, wherein the glutamate- and/or aspartate-rich segment comprises 2-7 amino acids in length with 50% or more amino acids selected from Glu (E) and/or Asp (D) residues.

27. The composition of claim 26, wherein the glutamate- and/or aspartate-rich segment comprises EE or EEE.

28. The composition of claim 15, wherein the bioactive peptide is capable of binding to a bioactive factor.

29. The composition of claim 28, wherein the bioactive factor is BMP-2.

30. The composition of claim 29, wherein the bioactive peptide is a BMP-2 binding peptide.

31. The composition of claim 30, wherein the bioactive peptide comprises at least 50% sequence identity with TSPHVPYGGGS (SEQ ID NO: 1).

32. The composition of claim 30, wherein the binding sequence comprises TSPHVPYGGGS (SEQ ID NO:1).

33. The composition of claim 28, wherein the composition further comprises the bioactive factor.

34. The composition of claim 28, wherein the composition does not comprise the bioactive factor.

35. A method comprising administering a composition of one or claims 1-34 to a subject.

36. The method of claim 35, wherein the composition is administered to repair a bone or tissue injury or defect.

37. The composition of claim 1, comprising:
(a) peptide amphiphile nanofibers comprising a supramolecular assembly of:
(i) bioactive peptide amphiphiles comprising (A) a hydrophobic non-peptidic segment, (B) a structural peptide segment, (C) a charged segment, and (D) a BMP-2 binding peptide; and
(ii) diluent peptide amphiphiles comprising (A) a hydrophobic non-peptidic segment, (B) a structural peptide segment, and (C) a charged segment; 1
(b) a PEG-containing polymer component;
(c) a ceramic component comprising TCP or HA; and
(d) BMP-2.

38. The composition of claim 1, comprising:
(a) peptide amphiphile nanofibers comprising a supramolecular assembly of:
(i) bioactive peptide amphiphiles comprising (A) a hydrophobic non-peptidic segment, (B) a structural peptide segment, (C) a charged segment, and (D) a BMP-2 binding peptide; and
(ii) diluent peptide amphiphiles comprising (A) a hydrophobic non-peptidic segment, (B) a structural peptide segment, and (C) a charged segment;
(b) a PEG-containing polymer component; and
(c) a ceramic component comprising TCP or HA;
wherein the composition does not comprise BMP-2.

39. A method of promoting osteogenesis comprising administering to a subject the composition of claim 37 or 38.

40. A method of repairing a bone injury or defect in a subject comprising administering the composition of claim 37 or 38 to the subject.

41. A method of promoting arthrodesis comprising administering to a subject the composition of claim 37 or 38.

42. A method of promoting spinal fusion comprising administering to a subject the composition of claim 37 or 38.


SEQUENCE LISTING (7 sequences)

SEQ ID NO: 1 - protein, 11 aa, Synthetic construct
TSPHVPYGGGS

SEQ ID NO: 2 - protein, 4 aa, Synthetic construct
AAVV

SEQ ID NO: 3 - protein, 6 aa, Synthetic construct
AAAVVV

SEQ ID NO: 4 - protein, 4 aa, Synthetic construct
VVAA

SEQ ID NO: 5 - protein, 6 aa, Synthetic construct
VVVAAA

SEQ ID NO: 6 - protein, 19 aa, Synthetic construct
TSPHVPYGGSEEEAAVVVK

SEQ ID NO: 7 - protein, 9 aa, Synthetic construct
EEEAAAVVV